(19) United States
(12) Patent Application Publication
(10) Pub. No.: US 2019/0125259 A1
(43) Pub. Date: May 2, 2019

(54) MULTIFUNCTIONAL INTELLIGENT WATCH CAPABLE OF MEASURING HEART BEAT RATE, BLOOD OXYGEN SATURATION, BLOOD PRESSURE, ELECTROCARDIOGRAM AND HAVING POSITIONING AND COMMUNICATION FUNCTIONS

(71) Applicant: Hsin Ming HUANG, Kaohsiung City (TW)

(72) Inventor: Hsin Ming HUANG, Kaohsiung City (TW)

(21) Appl. No.: 15/835,002

(22) Filed: Dec. 7, 2017

(30) Foreign Application Priority Data
Oct. 31, 2017 (TW) 106216144

(51) Int. Cl. A61B 5/00; A61B 5/024; A61B 5/0205

(52) U.S. Cl. CPC A61B 5/681; A61B 5/02438; A61B 5/021; A61B 5/02444; A61B 5/0002; A61B 5/0205

(57) ABSTRACT

A multifunctional intelligent watch includes a watch case and a main control motherboard. More than one conductive electrodes, transceivers and a SIM card slot are disposed on the watch case. The conductive electrodes and the transceivers form a current circuit for detecting and measuring the collected data. The main control motherboard serves to calculate the relevant data to display the data on a screen of the watch face. The data are uploaded to the cloud via a third-party APP of the intelligent mobile phone. Through the cloud computing technology, the complete data such as the electrocardiogram can be shown. A specific computing technology is used to precisely catch abnormal electrocardiogram and transmit an abnormal notice. Accordingly, a user can conveniently obtain many diagnostic data and automatically get an alert of any abnormal detected value. The SIM card serves to position and transmit data and provide communication function to ensure safety.

### BACKGROUND OF THE INVENTION

### 1. Field of the Invention

The present invention relates generally to a multifunctional intelligent watch capable of measuring heart beat rate, blood oxygen saturation, blood pressure, electrocardiogram and having positioning and communication functions, and more particularly to a portable multifunctional watch (or bracelet), which can be conveniently used.

### 2. Description of the Related Art

In general, the physiological parameters are respectively measured by different measurement implements. For example, the blood pressure is measured by a blood-pressure meter, the heart beat rate is measured via an electrocardiogram, while the blood oxygen saturation is obtained by a pulse oximeter. Each physiological parameter is measured by a specific implement that has only one function. This is quite time-consuming and inconvenient. Especially, with respect to those patients who need to measure the heart beat rate and blood pressure at any time, the conventional measurement implement has a huge volume and only one single function so that it is impossible for the patient to carry the measurement implement with him/her all the time to readily measure the physiological parameter. In order to solve the above problem, a wearable measurement device has been developed on the market. However, the currently commercially available wearable measurement implement capable of measuring the physiological parameters only has the functions of measuring heart beat pulse rate, step number, consumption of calorie, etc., while there is rarely any wearable measurement implement capable of measuring and analyzing deeper or more information concerning pulse and health. In addition, such wearable measurement implement can hardly sufficiently manage the healthy of personal cardiovascular system or analyze the physical/mental pressure. Therefore, the current wearable measurement implement cannot provide real-time physiological data detection and measurement. Moreover, in case an elder is lost and the elder is not used to carry a mobile phone, the lost elder can be hardly located. This will put the life of the elder in danger.

### SUMMARY OF THE INVENTION

It is therefore a primary object of the present invention to provide a multifunctional intelligent watch capable of measuring heart beat rate, blood oxygen saturation, blood pressure, electrocardiogram and having positioning and communication functions. The multifunctional intelligent watch includes a watch case and a main control motherboard. More than one conductive electrodes, transceivers and a SIM card slot are disposed on the watch case. The conductive electrodes and the transceivers form a current circuit for detecting and measuring the collected data. The main control motherboard serves to calculate the relevant data. The data are uploaded to the cloud via a third-party APP of the intelligent mobile phone. Through the cloud computing technology, the data such as heart beat rate, blood oxygen saturation, blood pressure and the electrocardiogram can be shown on the display unit of the watch case. Accordingly, the multifunctional intelligent watch can be used to conveniently, quickly and precisely measure various physiological parameters. In addition, the SIM card inserted in the SIM card slot can provide positioning and communication functions to directly upload data and help in locating a lost elder and communicating with the elder.

To achieve the above and other objects, the multifunctional intelligent watch capable of measuring heart beat rate, blood oxygen saturation, blood pressure, electrocardiogram and having positioning and communication functions includes a watch case and a main control motherboard. The watch case has a watch face and a back face. A display unit is disposed on the watch face. In addition, more than one conductive electrodes, transceivers capable of emitting red light, a speaker and a microphone are disposed on the watch case. More than one conductive electrodes, transceivers capable of emitting green light and a SIM card slot are disposed on the back face. The conductive electrodes and the transceivers form a current circuit for detecting and measuring the relevant diagnostic values of the cardiovascular system. The watch is wiredly or wirelessly electrically connected to the main control motherboard. The main control motherboard is disposed in the watch case. The main control motherboard includes a calculation chip electrically connected to the conductive electrodes for receiving or calculating and analyzing the data detected and measured by the transceivers and upload the data to the cloud. Via the cloud computing technology, the information such as heart beat rate, blood oxygen saturation, blood pressure and electrocardiogram can be shown on the display unit. A SIM card can be inserted in the SIM card slot to provide positioning and communication functions so as to directly upload data and help in locating a lost elder and finding the elder.

The present invention can be best understood through the following description and accompanying drawings, wherein:

### BRIEF DESCRIPTION OF THE DRAWINGS

FIG. 1 is a perspective view of the present invention;

FIG. 2 is a front view of the present invention;

FIG. 3 shows the use of the present invention;

FIG. 4 shows that the use of the present invention is completed;

FIG. 5 is a view of another embodiment of the present invention, in which the conductive electrodes are disposed in different positions;

FIG. 6 is a back view of the other embodiment of the present invention;

FIG. 7 shows the use of the other embodiment of the present invention;

FIG. 8 is a view of still another embodiment of the present invention, in which the conductive electrodes are disposed in different positions; and

FIG. 9 shows the use of the other embodiment of the present invention.

### DETAILED DESCRIPTION OF THE PREFERRED EMBODIMENTS

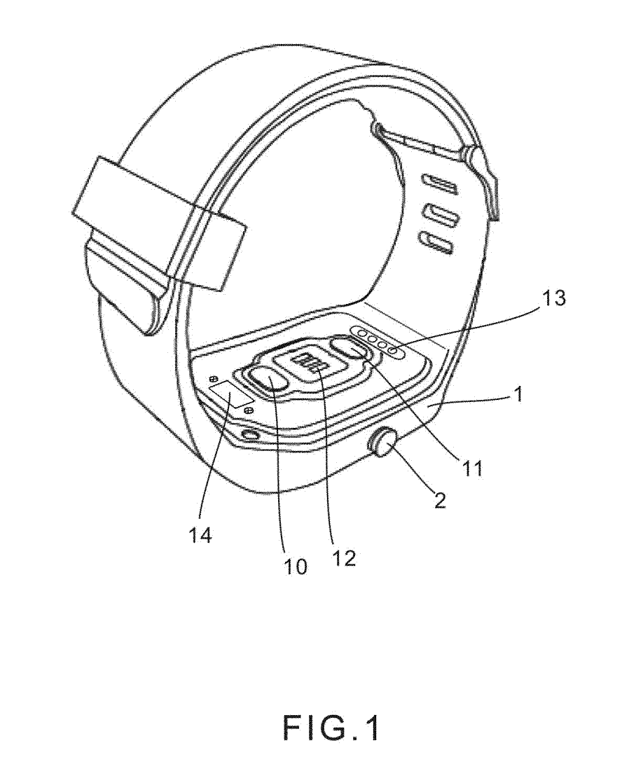

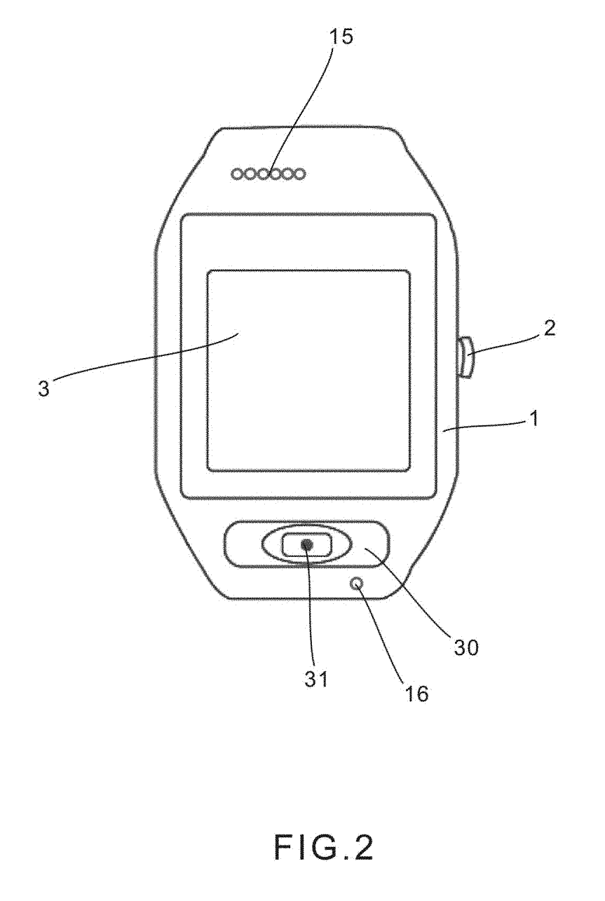

Please refer to FIGS. 1 and 2. The multifunctional intelligent watch capable of measuring heart beat rate, blood oxygen saturation, blood pressure, electrocardiogram and having positioning and communication functions of the present invention includes a watch case 1 and a main control motherboard (not shown) disposed in the watch case 1. Two conductive electrodes 10, 11 are disposed on the back face of the watch case 1. A transceiver 12 capable of emitting green light is disposed between the two conductive electrodes 10, 11. A charger socket 13 is disposed behind the transceiver 12 for connecting with a charger to charge the watch. A SIM card slot 14 is disposed in front of the transceiver 12 for a SIM card to insert therein so as to transmit data and provide positioning communication effect. The SIM card can directly upload monitoring data to help in obtaining the location of a lost elder. In addition, a pushbutton switch 2 is disposed on one side of the watch case 1 for activating the main control motherboard and the transceiver 12 to operate. A display unit 3 is disposed on the watch face of the watch case 1. A conductive electrode 30 is disposed under the display unit 3. The conductive electrode 30 is equipped with a transceiver 31 capable of emitting red light for measuring the blood oxygen saturation. Moreover, a speaker 15 and a microphone 16 are arranged on the front face of the watch case 1 for communication.

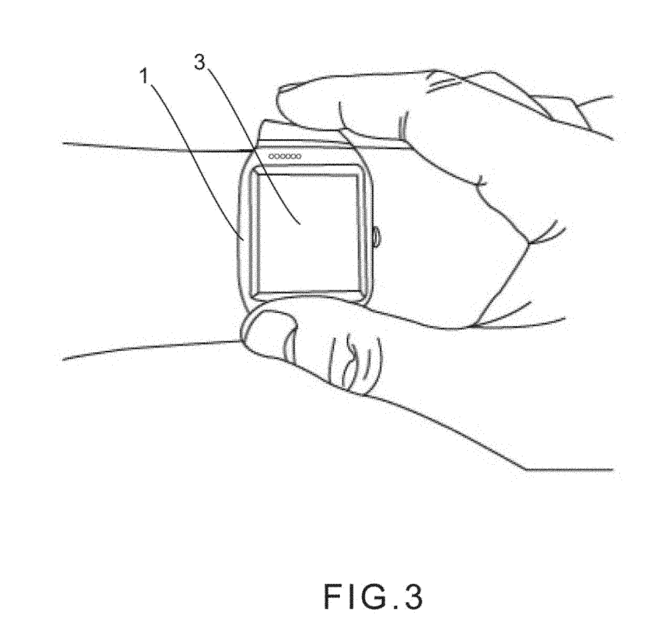

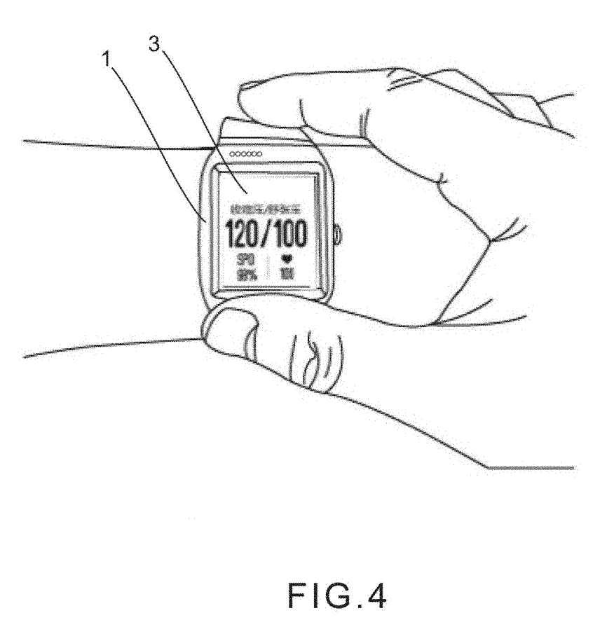

In first use (as shown in FIG. 3), a user wears the watch case 1 on his/her wrist with the two conductive electrodes 10, 11 and the transceiver 12 capable of emitting green light attached to the skin of the wrist. The pushbutton switch on one side of the watch case 1 is pressed for a long time to start the watch for detection and measurement. After the display unit 3 enters the recording page, the thumb of the right hand attaches to the metal electrode area to trigger the transceiver capable of emitting red light to lastingly emit red light so as to start one time of healthy recording. At this time, the page of the watch will show the data (as shown in FIG. 4). When the transceiver capable of emitting red light is turned off, this means the recording is ended. In case the recorded values are normal, the result will be directly displayed. In case there are abnormal values, the watch will automatically indicate the abnormality.

In case an elder is lost and the elder is not used to carry a mobile phone, the SIM card inserted in the SIM card slot 14 can provide positioning and communication function as a real-time monitoring device to directly transmit monitoring data. Accordingly, it is unnecessary to use the mobile phone to upload data. Also, the watch can be used to find a lost elder and locate the elder.

With respect to the measurement method of the present invention, a sensor capable of emitting green light is used to measure the pulse rate and a sensor capable of emitting red light is used to measure the blood oxygen saturation. The blood flow transferred through the artery is regularly varied with the pumping period of the heart. Therefore, by means of measuring the change of the blood flow of a specific part of the body, the pulse rate can be obtained from the periodicity of the measured signal.

Furthermore, when light beam passes through a human body, the light beam will be absorbed by different tissues and decayed. The volume of the blood in the vessel is obviously varied with the heart beat. The periodical change of volume leads to different decay amount. Therefore, when light beam penetrates through the tissues of the skin, by means of observing the decay of light intensity, a waved diagram with periodical heaving and dipping can be obtained. The interval time between the wave peaks is divided by 60 to obtain the heart beat number per minute.

With respect to the reading of the blood oxygen saturation, the light beam projected onto the skin is absorbed by the blood in the vein or artery or reflected back to the detector. The composition of signal variation is in consistence with the artery blood flow of the synchronous heart beat change. The period of the signal change indicates the pulse rate. The ratio of the minimum detector signal to the maximum detector signal provides a basis for the judgment of blood oxygen saturation. Concerning the formation of the electrocardiogram, the conductive electrodes respectively disposed on the bottom and surface of the watch case are used. The conductive electrode of the bottom is in contact with the wrist, while another finger touches the conductive electrodes of the watch face to form the current circuit for electrocardiogram measurement so as to measure the curve of the electrocardiogram. With respect to the measurement of blood pressure, the transmission speed of the pulse is directly related to the blood pressure. When the blood pressure is high, the pulse wave is transmitted fast, while when the blood pressure is low, the pulse wave is transmitted slowly. The pulse wave transmission time and speed can be obtained via the electro-cardiac signal and the pulse wave signal plus some regular body parameters (such as height and weight) to further calculate out the blood pressure value.

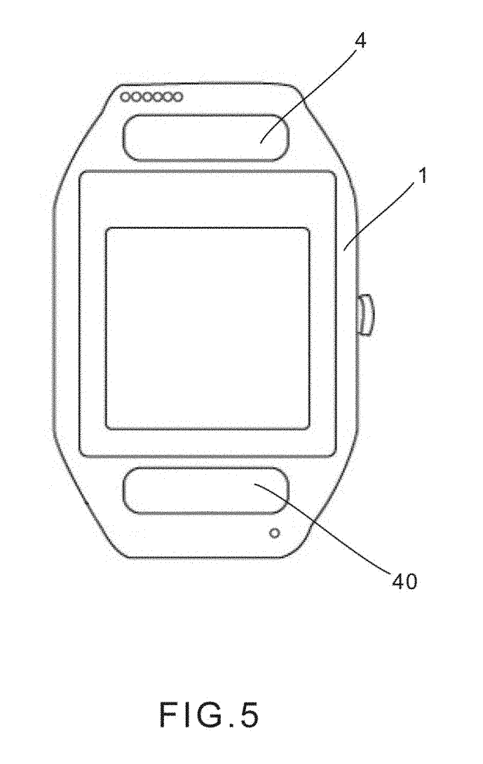

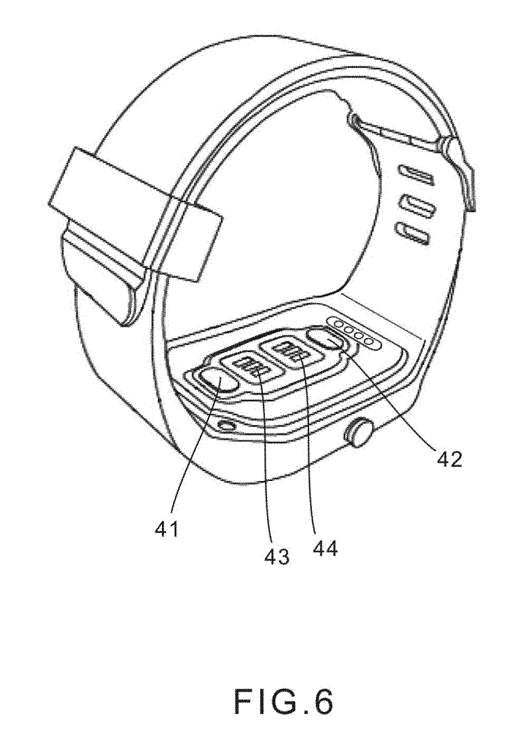

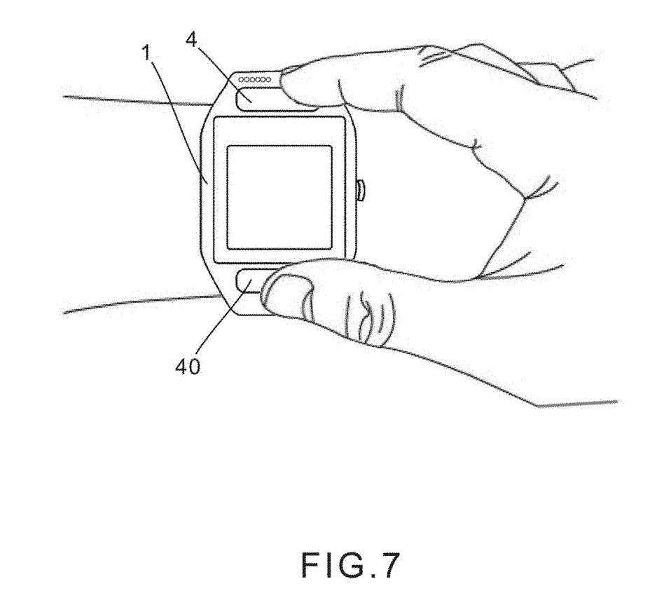

Please now refer to FIGS. 5, 6 and 7, which show another embodiment of the present invention. In the other embodiment, the multifunctional intelligent watch of the present invention includes a watch case 1 and a main control motherboard (not shown) disposed in the watch case 1. Two conductive electrodes 4, 40 are respectively disposed on the upper and lower sides of the watch face of the watch case 1. The two conductive electrodes 4, 40 are electrically connected to the main control motherboard. Also, two conductive electrodes 41, 42 are disposed on the back face of the watch case 1. A transceiver 43 capable of emitting red light and a transceiver 44 capable of emitting green light are disposed between the two conductive electrodes 41, 42 (as shown in FIG. 6). Two fingers touch the two conductive electrodes 4, 40, whereby the conductive electrodes and the transceiver form a current circuit for detecting and measuring the relevant diagnostic values of the cardiovascular system.

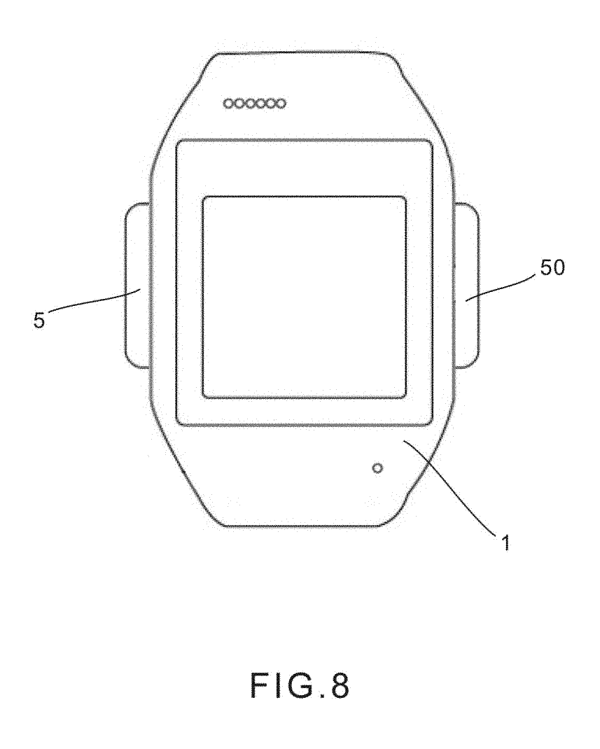

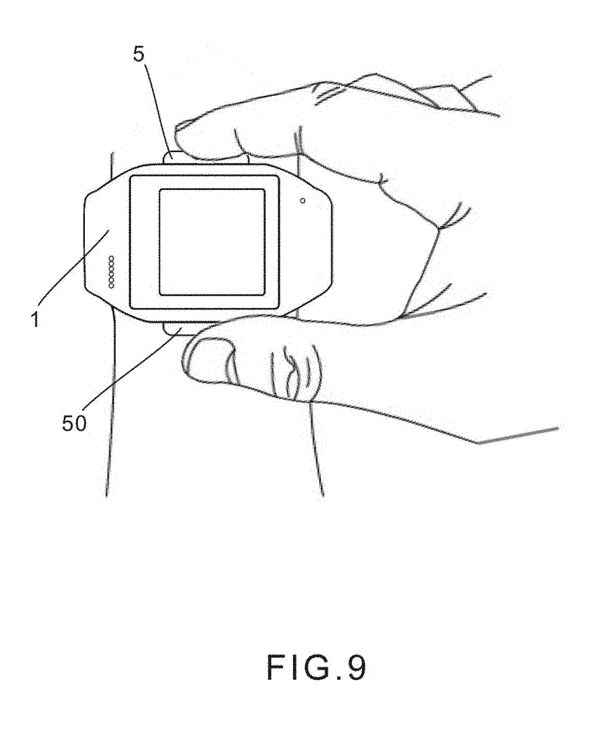

Please further refer to FIGS. 8 and 9, which show still another embodiment of the present invention. In this embodiment, the multifunctional intelligent watch of the present invention includes a watch case 1 and a main control motherboard (not shown) disposed in the watch case 1. Two conductive electrodes 5, 50 are respectively disposed on two sides of the watch face. The two conductive electrodes 5, 50 are electrically connected to the main control motherboard. Two fingers touch the two conductive electrodes 5, 50 (as shown in FIG. 9), whereby the conductive electrodes and the transceiver form a current circuit for detecting and measuring the relevant diagnostic values of the cardiovascular system.

The above embodiments are only used to illustrate the present invention, not intended to limit the scope thereof. Many modifications of the above embodiments can be made without departing from the spirit of the present invention.

What is claimed is:

1. A multifunctional intelligent watch capable of measuring heart beat rate, blood oxygen saturation, blood pressure, electrocardiogram and having positioning and communication functions, comprising:
a watch case having a watch face and a back face, a display unit being disposed on the watch face, more than one conductive electrodes, transceivers capable of emitting light, a speaker and a microphone being disposed on the watch case for a finger to touch to transmit signals, more than one conductive electrodes, transceivers capable of emitting light and a SIM card slot being disposed on the back face to attach to a wrist for emitting light and receiving the light reflected back from the skin and vessel to analyze the obtained pulse wave signals, the SIM card inserted in the SIM card slot serving to provide communication and positioning functions and uploading data to help in finding a lost elder and obtaining the location of the elder; and
a main control motherboard including a calculation chip electrically connected to the conductive electrodes for receiving the detected and measured data transmitted from the conductive electrodes, whereby the conductive electrodes and the transceivers form a current circuit for detecting and measuring the collected data, the main control motherboard serving to calculate the data and upload the data to the cloud computing technology and the display unit serving to display the information of heart beat rate, blood oxygen saturation, blood pressure and electrocardiogram, the SIM card serving to provide communication and positioning functions.

2. The multifunctional intelligent watch capable of measuring heart beat rate, blood oxygen saturation, blood pressure, electrocardiogram and having positioning and communication functions as claimed in claim 1, wherein the conductive electrodes are disposed on upper and lower sides of the watch face of the watch case.

3. The multifunctional intelligent watch capable of measuring heart beat rate, blood oxygen saturation, blood pressure, electrocardiogram and having positioning and communication functions as claimed in claim 1, wherein the conductive electrodes are disposed on two lateral sides of the watch case.
